(19) United States
(12) Patent Application Publication
(10) Pub. No.: US 2023/0026700 A1
(43) Pub. Date: Jan. 26, 2023

(54) System and Method for Performance Prediction Based on Resting-State Electroencephaloraphy

(71) Applicants: Arash Mahyari, Pensacola, FL (US); Toshiya Miyatsu, Pensacola, FL (US); Peter Pirolli, Pensacola, FL (US)

(72) Inventors: Arash Mahyari, Pensacola, FL (US); Toshiya Miyatsu, Pensacola, FL (US); Peter Pirolli, Pensacola, FL (US)

(21) Appl. No.: 17/668,616

(22) Filed: Feb. 10, 2022

Related U.S. Application Data
(60) Provisional application No. 63/147,811, filed on Feb. 10, 2021.

(51) Int. Cl. A61B 5/372; A61B 5/00

(52) U.S. Cl. CPC A61B 5/372; A61B 5/0042

(57) ABSTRACT

A system and method comprising several hardware and software components that work together to achieve the goal of performance prediction. The hardware components include neuroimaging collection hardware and a computing system. The neuroimaging hardware obtains the brain signals and sends this first set of data to a computational resource for further analysis. A second sent of data is the task performance scores of the participants. This second set is also set to the computational resource. The inventive system can predict a learning rate of target tasks for an individual from just a few minutes of off-task, resting-state neuroimaging data.

### CROSS-REFERENCES TO RELATED APPLICATIONS

This non-provisional patent application claims priority to U.S. Provisional App. No. 63/147,811. The parent application listed the same inventors. It was filed on Feb. 10, 2021.

### STATEMENT REGARDING FEDERALLY SPONSORED RESEARCH OR DEVELOPMENT

Funding for this invention was provided by a contract from the Defense Advance Research Projects (DARPA) Biological Technologies Office (BTO) under its Measuring Biological Aptitude (MBA) Program.

### MICROFICHE APPENDIX

Not Applicable

### BACKGROUND OF THE INVENTION

### 1. Field of the Invention

The present invention pertains to the field of human performance prediction. More specifically, the invention comprises a system and method for predicting target task performance using just a few minutes of off-task, resting-state electroencephalography (EEG).

### 2. Description of the Related Art

A common method of personnel selection is to test the candidates on the task that the recruited position requires to perform. For example, the military may test its candidates on marksmanship, and the U.S. Federal Aviation Administration may test its candidates on air traffic control. However, an inherent challenge associated with this approach is that testing task performance is costly. Evaluators need to secure the space and the required equipment, and both testing and the analysis of the collected task performance data take time and labor.

Research in the past couple of decades—driven by the advances in neuroimaging techniques—has revealed that the human brain at rest is not a dormant organ waiting for the next commands. Rather, the regions of the brain that regularly work together to accomplish tasks exhibit synchronized fluctuation in their activities even at a resting state, a phenomenon known as the functional connectivity (Van Den Heuvel & Pol, 2010). Further, recently developed machine learning-based, high fidelity analysis can detect the neural networks that have been strengthened as a result of training of specific cognitive systems within individuals, and these measures can be used to predict a variety of task performance (e.g., Gong, et al., 2017; Rogala, et al., 2020). For instance, the inventors' own analysis of an electroencephalogram (EEG) dataset published elsewhere (Rogala, et al., 2020) with the current method described in this application showed that the resting-state connectivity reliably predicted participants' performance in a shooting task (Mahyari, et.al., 2022, Phase synchrony measures from resting-state EEG reading predicts shooting performance and future learning). In addition, the application of the same inventive method to recently acquired in-house EEG data showed that the resting-state connectivity illuminated by this method also predicted performance in a shooting simulator, a semantic memory task (verbal fluency task), and visuo-spatial tracking performance (Neurotracker) (Mahyari, et.al. 2022, Resting-State EEG as a Multidimensional Aptitude Assessment Tool).

A core idea of the present inventive system is to leverage these emerging techniques and build a system that predicts target task performance that users are interested in measuring. The users first input the resting-state neuroimaging data, including but not limited to data acquired from EEG, functional magnetic resonance imaging (fMRI), positron emission tomography (PET), magnetoencephalography (MEG), and near infrared spectroscopy (fNIRS), as well as candidates' task performance. After enough initial inputs (e.g., 30 data points), the algorithm becomes capable of computing the relationship between candidates' neural configuration and the task performance. Thereafter, whenever users input candidates' resting-state neuroimaging data, the algorithm gives a prediction on the candidates' target task performance.

There is prior work regarding the use of neuroimaging for prediction purposes, but it is quite limited. WIPO Pub. No. WO 2016/029293 (Grass and Ghadrigolestani) describes the use of EEG data to predict epileptic seizures. U.S. Pat. No. 8,521,270 (Hunter and Leuchter) describes the use of EEG data to predict a patient's response to certain medications. The present invention more broadly applies the use of neuroimaging data to predict human performance.

### BRIEF SUMMARY OF THE INVENTION

The present inventive system and method comprises several hardware and software components that work together to achieve the goal of performance prediction. The hardware components include neuroimaging data acquisition systems and a computing system. The neuroimaging data acquisition system obtains the brain signals and sends this first set of data to a computational resource for further analysis. A second sent of data is the task performance scores of the participants. This second set is also sent to the computational resource. The inventive system can predict a learning rate of target tasks for an individual from just a few minutes of off-task, resting-state neuroimaging data.

Additional objects and advantages of the present invention are as follows:

1. The invention can use phase synchrony for extracting features from resting state neuroimaging data (EEG, fMRI, etc.).

2. The invention can select significant phase synchrony values correlated with the target task.

3. The invention can predict the performance of the task (score) from the selected phase synchrony values.

4. The invention can use linear regression algorithms to make score predictions (such as a numerical score of 0 to 100).

5. The invention can use logistic regression algorithms to make classification predictions (such as pass or fail).

6. The invention can use deep learning neural networks to learn features and predict performance scores from resting-state neuroimaging data.

7. The invention can use graph-based theory to build graphs from the phase synchrony values, analyze the graphs, and predict the performance score.

8. After accumulating enough data (such as resting state neuroimaging data and task performance data), the invention can predict task performance from neuroimaging data without having any additional task performance data (without having candidates perform the task).

### BRIEF DESCRIPTION OF THE SEVERAL VIEWS OF THE DRAWINGS

FIG. 1 is a schematic view depicting the data gathering and computation steps.

FIG. 2 is a schematic view, depicting the operations of the computing system.

FIG. 3 is a schematic view, depicting the gathering of data sets from diverse sources and the return of results to those sources.

FIG. 4 is a graphical view, showing exemplary predictions made by the inventive system.

### DETAILED DESCRIPTION OF THE INVENTION

The inventive system consists of several hardware and software components that work together to achieve the goal. The hardware components include a neuroimaging device, such as an EEG headset (a headset with incorporated EEG electrodes), and a computing system. The neuroimaging device obtains the brain signals and sends it to the computational resource for further analysis.

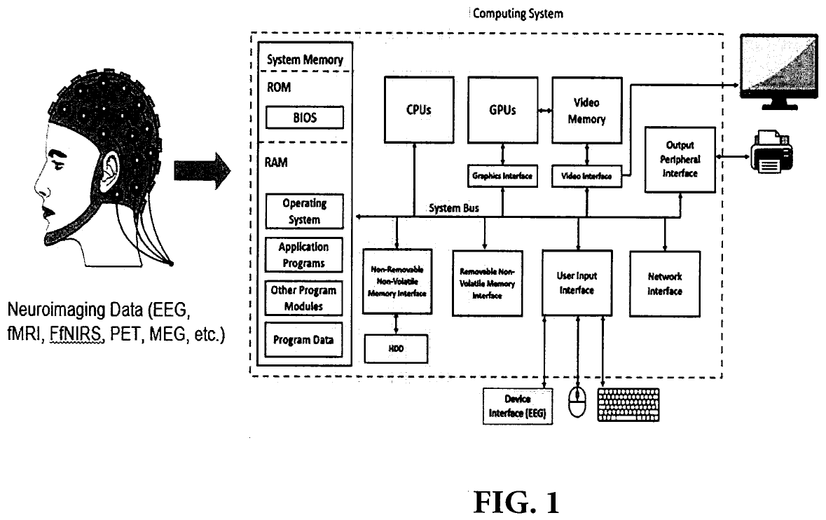

The inventive system collects two types of data sets. The first dataset is the neuroimaging signals collected from the participants, including but not limited to EEG, fMRI, fNIRS, PET, or MEG signals collected from the participants. The second set of data is the task performance scores of the participants. The collections and processing of this data is depicted in FIG. 1.

The computation resource passes the neuroimaging data through several modules to process and analyze the data. To improve the performance and the accuracy of the proposed system, the inventors use an ensemble of methods in the computing system. Each of the three methods (phase synchrony, deep learning, and graph-based analyses) provides a separate score for the performance that will be used to evaluate the participants.

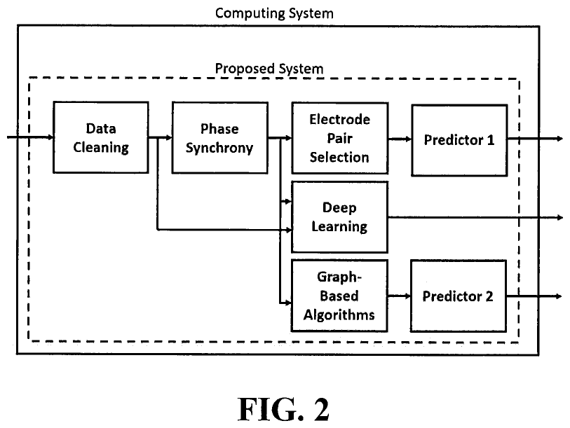

The modules present within the computing processes are depicted in FIG. 2. Each of these functional blocks is described in more detail in the following:

1. Data Cleaning: The recorded EEG data is often noisy. Several data cleaning procedures apply to the raw EEG signals to prepare them for processing: Data averaging, principal component analysis, and Current Source Density using Laplacian transformation;

2. Phase Synchrony: The proposed system uses the phase synchrony method to calculate the interaction among the brain regions. Phase synchrony is the feature extraction step of the proposed system. The phase synchrony quantifies the intensity of the interaction between brain regions. After calculating the phase synchrony, the values are scaled between zero and one using phase-locked value (PLV). One means perfectly synchronized, and zero means the brain regions are not synchronized at all;

3. Brain Connectivity Feature Selection: After calculating the phase synchrony among pairs of brain regions (e.g., regions of interest (ROIs) in fMRI and PET, or electrodes in EEG), the proposed system will automatically identify the significant pairs (phase synchrony values) correlating with the performance scores. The inventors have used several steps to identify significant pairs. The inventors used the stepwise algorithm with different criteria (e.g., AIC, BIC) to select the variables that are significantly correlated with the performance scores. The inventive method further down-selects the pairs selected by the stepwise algorithm based on their correlation score. The invention selects only pairs that have high correlation coefficients between performance score and the electrode pair PLV;

4. Predictor 1 and 2: The selected pairs are used to train predictor models. The proposed system consists of two components: one classifier (logistic regressors) and one linear regressor. The classifier is used to fit a logistic regression model to the training data. The classifier indicates whether the participant is going to perform exceptionally well during the task (e.g., above 80 percentile) or whether they pass or fail the task when such distinction is available. The linear regressor predicts the performance score based on the PLVs of the selected pairs;

5. Deep Learning: Once an appropriate amount of data have been collected, the data is used to train a deep neural network. The input of the deep neural network module is from the cleaned data as well as phase synchrony. The deep neural network module consists of several convolutional and fully connected layers. On the one hand, the deep neural network is able to extract complex, hidden patterns from the input data. In the inventive system, the inventors leverage this capability of deep networks to extract hidden patterns from the cleaned neuroimaging data. On the other hand, neural networks are not able to extract the pairwise synchronization within the neuroimaging data as well as the analytical approaches described in the Steps 2-4 above. Thus, the inventors use the phase synchrony as the input to the deep neural network module, and it presents an improvement upon the previous method employing the phase synchrony or the deep learning approach alone; and

6. Graph-Based Algorithms: This module will use the calculated phase synchrony to construct a graph. The nodes of the graph are the ROIs of fMRI/PET or electrodes of the EEG headset, and the edges are the values of the phase synchrony. The graph theory is used to extract several characteristics from the graph. These characteristics are clustering coefficient, path length, global efficiency, local efficiency. These characteristics are used as the input to the second predictor to predict the performance score.

Another important problem related to personnel selection and assignment is that evaluators ultimately want to select candidates who will be best at the target task over time (e.g., after training) not the candidates who only perform well at the time of selection. Predicting who will perform well after training that has not yet been given is traditionally difficult. The present invention addresses these challenges by building a system that can predict learning rates for target tasks from just a few minutes of off-task, resting-state neuroimaging.

The inventive system achieves its objectives by:

1. Using phase synchrony for extracting features from resting-state neuroimaging data (EEG, fMRI, etc.);

2. Selecting significant phase synchrony values correlated with the target task;

3. Predicting whether the performance score will improve if the subject receives proper training—using the selected phase synchrony values;

4. Using linear regression algorithms to make learning rate predictions (e.g., score of 0-100);

5. Using logistic regression algorithms to make classification predictions (e.g., above 75 percentile learner or not);

6. Using deep learning neural networks to learn features and predict whether the performance score will improve if the subject receives proper training from resting-state neuroimaging data (EEG, fMRI, etc.);

7. Using graph-based theory to build graphs from phase synchrony values, analyze graphs, and predict whether the performance score will improve if the subject receives proper training; and

8. After accumulating enough data (i.e., resting-state neuroimaging data & pre-post training task performance data), the algorithm in the present invention predicts task learning rates from neuroimaging data alone (without having candidates perform the actual task).

The inventors have discovered in a recent analysis of a dataset published elsewhere (Rogala, et al., 2020) that the resting-state neural connectivity reliably predicted participants' learning rate in a shooting task. The algorithm was able to predict the above average learners (i.e., above 50 percentile in shooting skill improvement after 2 months of training supervised by a professional coach) at over 80% accuracy (Mahyari, et al., 2022). A significant idea in the proposed invention is to leverage these emerging techniques and build a system that predicts future learning rate in target tasks that users are interested in measuring.

The users of the inventive system first input the resting-state neuroimaging data as well as candidates' initial task performance score and the post-training performance score. After enough initial inputs (e.g., 30 data points), the algorithm becomes capable of computing the relationship between candidates' neural configuration and the learning rate (i.e., post-training performance score minus initial task performance score). Thereafter, whenever users input candidates' resting-state neuroimaging data, the algorithm gives a prediction on the candidates' learning rate.

The system architecture of the learning rate prediction system is identical to the task performance prediction system disclosed in Pub. No. WO2016/0292293. However, users input the post-training task performance score in addition to the initial task performance score along with the neuroimaging data, so that the learning rate can be computed.

Another common approach to personnel selection and assignment is to have candidates complete a variety of general cognitive and personality aptitude tests, such as an intelligence test. However, testing each candidate with several of these tests is costly. Each test takes time, effort, and financial cost to administer, and it is fatiguing for the candidates to complete many tests. In addition, evaluators often have to rely on the candidates' self-report assessment of themselves, and these self-report assessments in the context of selection and assignment can be unreliable because the candidates often answer according to what they think would be desirable to get the position instead of truthfully answering, or because candidates themselves might not have accurate understanding of their own traits. For example, research on the Myers-Briggs Type Indicator, one of the most popular assessments for job assignment at the workplace based on self-report personality testing, showed that the test is largely ineffective in predicting people's success at various jobs (Gardner & Martinko, 1996; Pittenger, 1993).

A central idea of the proposed work is to leverage these emerging techniques and build a system that estimates the scores on common cognitive and personality tests. The users first input the resting-state neuroimaging data as well as candidates' scores on the cognitive/personality tests of interest. After enough initial inputs (e.g., 30 data points), the algorithm becomes capable of computing the relationship between candidates' neural configuration and the test scores. Thereafter, whenever users input candidates' resting-state neuroimaging data, the algorithm gives an estimation of candidates' test scores.

Yet another important problem related to personnel selection and assignment is that evaluators often have a collection of task performance and general traits they want to test. For example, the military may want to know candidates' ability in marksmanship, navigation, and communication, in addition to general cognitive and personality traits. However, testing for all of these diverse task performance and cognitive/personality traits would be unrealistic from the time and financial resource standpoint.

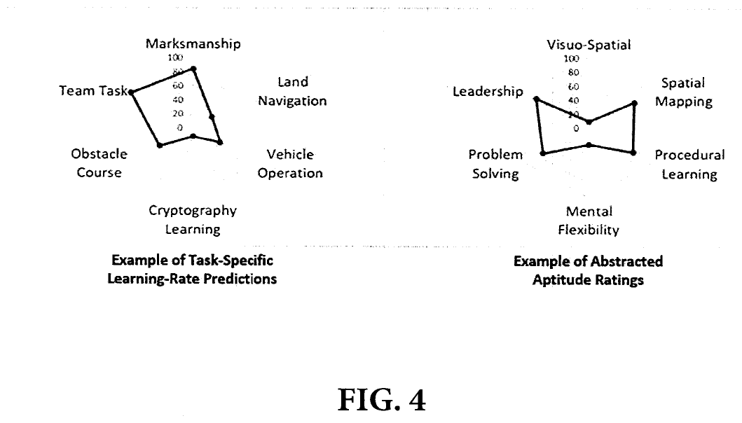

The present invention addresses these challenges by building a centralized system based on Pub. No. WO2016/0292293 and U.S. Pat. No. 8,521,270 that can ultimately give a profile of aptitude assessment based on candidates' neural configurations observed through resting-state neuroimaging. The proposed system ultimately gives an aptitude rating (e.g., 0-100) for various target task types (see FIG. 4 for examples). Specifically, the learning rate system disclosed in U.S. Pat. No. 8,521,270 is able to predict how well the candidate will perform in the target task for which task performance data have been inputted. In the present inventive system, the inventors extend the capability of the '270 Patent to predict the learning rate of the candidate for multiple tasks simultaneously through the use of the previous data accumulated in the central data repository. For example, the system will provide the learning rates for shooting, language learning, land navigation, parachuting, etc. The user, then, is able to see which candidates are best suited for particular tasks. In addition, this system gives estimated scores of several cognitive and personality tests whose data have previously been collected through the patent 3 above and stored in the central data repository.

The inventive system takes data inputs (neuroimaging data accompanied by task performance, learning rate, and cognitive/personality tests) from all previous users testing for various tasks/tests and stores them in a central data repository. Based on these accumulated data, the system creates a profile of neural characteristics (key brain connections, graph-based metrics, etc.) suited for each task performance and predictive of cognitive/personality traits over time. When new users input their candidates' neuroimaging data, the system then gives prediction and estimation of a collection of task performance, learning rate, and cognitive/personality traits.

The proposed invention uses a centralized system that takes data inputs from users, gives predicted learning rate for various tasks as output, and improves the accuracy of the prediction and expands the types of tasks for which predictions can be made over time through accumulation and continuous re-analysis of the data.

Data input & Learning Rate Prediction: The proposed system collects the same two types of data sets as described in U.S. Pat. No. 8,521,270. The first dataset is the neuroimaging signals collected from the participants, and the second set of data is the task performance scores of the participants before and/or after training. The same algorithm is used to derive learning rate predictions.

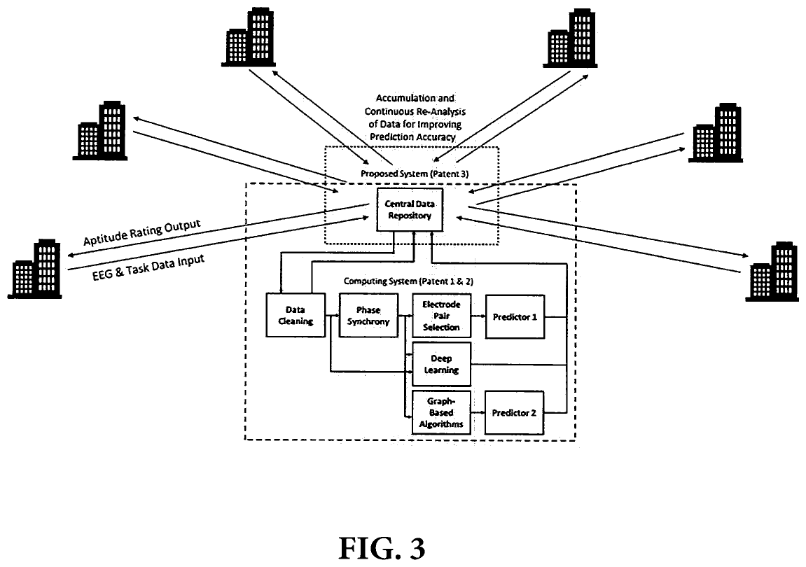

Centralized Algorithm Development and Refinement: The system stores each set of data (neuroimaging and task performance) in the central data repository (see FIGS. 3 and 4). Importantly, the proposed system draws from all the accumulated data to derive learning rate predictions for various tasks. Accumulation of data of the same task over time (e.g., shooting prediction from user A and user B or user A's first and second sets of data) makes the prediction more accurate while the accumulation of data of similar tasks will allow abstraction of the task-specific prediction to assessment of the underlying cognitive traits.

Once the neuroimaging data is collected and the task performance data is collected, the inventive system creates a profile of neural characteristics (key brain connections, graph-based metrics, etc.) suited for each task performance and predictive of cognitive/personality traits.

The system uses phase synchrony for extracting features from resting-state neuroimaging data (EEG, fMRI, etc.).

The system uses graph-based theory to build graphs from the computed phase synchrony values, analyze graphs, and extracts graph-based features.

The system matches the extracted phase synchrony and graph-based features for a given candidate with the features associated with the accumulated task performance, learning rate, and cognitive/personality test scores and give comprehensive aptitude scores (see FIG. 3 for a conceptual example).

Traditional aptitude and psychological testing first identified the psychological construct of interest (e.g., visuo-spatial learning ability) and then constructed a task or questionnaire items that measured that construct. This prior art method, by definition, is capable of measuring only that psychological construct that the test is intended to measure. The proposed system, in contrast, identifies the brain connectivity characteristics associated with various aptitude and psychological constructs first through accumulation of neuroimaging and performance data, and then matches the brain characteristics of a given candidate with the accumulated data to simultaneously give various task performance, learning rate, and cognitive/personality test scores.

The inventive system is a centralized system that stores data inputs from all previous users, takes new neuroimaging data as input, and gives comprehensive aptitude scores (predicted task performance, learning rate, and cognitive/personality traits) as output. The system improves the accuracy of the prediction and expands the types of tasks and tests for which predictions can be made over time through accumulation and continuous re-analysis of the data.

Preferred embodiment include the following features:

1. Data accumulation: The proposed system collects two data sets. The first dataset is the neuroimaging data collected from the participants, and the second set of data is the task performance, learning rate, and cognitive/personality tests.

2. Centralized algorithm development and refinement: The system stores each set of data (e.g., neuroimaging and task performance) in the central data repository. Importantly, the proposed system draws from all the accumulated data to derive the predictions for various tasks. Accumulation of data of the same task over time (e.g., shooting prediction from user A and user B or user A's first and second sets of data) makes the prediction more accurate while the accumulation of data of different tasks will expand the tasks and traits for which the system gives its aptitude rating.

The preceding description contains significant detail regarding the novel aspects of the present invention. It should not be construed, however, as limiting the scope of the invention but rather as providing illustrations of the preferred embodiments of the invention. Thus, the scope of the invention should be fixed by the claims ultimately drafted, rather than by the examples given.

Having described our invention, we claim:

1. A method for using neuroimaging data to predict performance, comprising:
(a) providing a computing system including a central processing unit and an associated memory;
(b) providing analytical software running on said central processing unit;
(c) collecting multiple sets of neuroimaging data for a first set of multiple human subjects and storing said neuroimaging data in said associated memory;
(d) collecting task performance data for said first set of multiple human subjects and storing said task performance data in said associated memory;
(e) using said analytical software to correlate said neuroimaging data to said task performance data, thereby creating a predictive model; and
(f) collecting multiple sets of neuroimaging data for a second set of multiple human subjects new human subjects; and
(g) using said predictive model to predict task performance for said second set of multiple human subjects.

2. The method for using neuroimaging data as recited in claim 1, wherein said neuroimaging data is selected from the group consisting of electroencephalography (EEG), functional magnetic resonance imaging (fMRI), positron emission tomography (PET), magnetoencephalography (MEG), and near infrared spectroscopy (fNIRS),

3. The method for using neuroimaging data as recited in claim 1, wherein said predictive model uses phase synchrony to calculate interaction among brain regions.

4. The method for using neuroimaging data as recited in claim 2, wherein said predictive model uses phase synchrony to calculate interaction among brain regions.
